(19) United States
(12) Patent Application Publication
(10) Pub. No.: US 2008/0312196 A1
(43) Pub. Date: Dec. 18, 2008

(54) Topical wart removal composition

(76) Inventor: Carl J. Cohen, Waterford, MI (US)

Correspondence Address: THE WEINTRAUB GROUP, P.L.C., Suite 240, 32000 Northwestern Highway, Farmington Hills, MI, 48334, US

(21) Appl. No.: 12/214,035

(22) Filed: Jun. 16, 2008

Related U.S. Application Data
(60) Provisional application No. 60/934,708, filed on Jun. 15, 2007.

(51) Int. Cl. A61K 31/60; A61P 17/12

(52) U.S. Cl. 514/163; 514/159

(57) ABSTRACT

A non-aqueous liquid composition for topical application to warts and other fibroids, papillomas, and other growths for the removal of same is defined by a non-aqueous solution of: (a) at least one keratolytic compound or agent, (b) an adhesive, (c) a dermal regeneration promoter, and (d) a solvent. The composition is applied to the growth or growth site on a daily basis for about at least seven days. The composition can be applied directly to the site or via an impregnated bandage.

### CROSS-REFERENCE TO RELATED APPLICATIONS

This application is a completion application of co-pending U.S. provisional patent application Ser. No. 60/934,708, filed Jun. 15, 2007, for “Topical Wart Removal Composition,” the entire disclosure of which is hereby incorporated by reference.

### BACKGROUND OF THE INVENTION

1. Field of the Invention

The present invention concerns compositions for topical application. More particularly, the present invention concerns liquid compositions for treating dermal conditions by topical application. Even more particularly, the present invention pertains to liquid compositions for promoting wart and other dermal growth removal.

2. Prior Art

As is known to those skilled in the art to which the present invention pertains, the removal of warts and other dermal growths affects nearly all persons. Warts and other dermal growths are typically removed either through excision or through chemical compositions used to dissolve or otherwise cause the dislodgement of same. Excision with a scalpel, scissors or the like is discomforting and may lead to scarring. On the other hand, heretofore known topical compositions may not be entirely efficacious, especially “over-the-counter” type compositions. Topically applied prescription compositions may create discomfort through burning, etc. Thus, the art consistently seeks out alternative topical compositions for removing skin growths, e.g. warts, such as that described hereinafter.

In providing such an alternative topical composition, it is highly desirable that it contain non-toxic pharmacological agents to preclude the necessity for licensure by regulatory agencies to thereby enable the product to get to the market in a much faster mode.

Heretofore many compositions with associated delivery devices have been proposed to remove warts and the like. For example, in U.S. Pat. No. 5,476,664, there is disclosed therein a plethora of treatments for the removal of warts. In particular, this patent teaches the utilization of anthralin as the active ingredient for wart removal. The patent further discloses that various other compositions have been used such as, for example, salicylic acid along with a polyacrylic delivery vehicle. Additionally, U.S. Pat. No. 4,016,264 teaches the use of a phosphonacetic acid for use in wart removal. Other relevant art can be found in the U.S. Pat. No. 5,618,850 which teaches the use of an alphahydroxy poly acid for wart removal. However, none of the prior art addresses the issue of skin regeneration at the location where the growth is removed. The present invention, as subsequently described overcomes this deficiency in the prior art.

### SUMMARY OF THE INVENTION

In accordance herewith, there is provided a non-aqueous liquid composition for topical application to warts and other fibroids, papillomas, and other growths for the removal of same.

The present composition, generally, comprises a non-aqueous solution of:

(a) at least one keratolytic compound or agent,

(b) an adhesive,

(c) a dermal regeneration promoter, and

(d) a solvent.

Adjuvants, such as perfumes and the like may be incorporated into the composition.

For a more complete understanding of the present invention reference is made to the following detailed description and accompanying example.

### DESCRIPTION OF THE PREFERRED EMBODIMENT

As hereinabove noted the present invention provides a non-aqueous liquid composition for topical application to warts and other fibroids, papillomas, and other growths for the removal of same, and which, generally, comprises:

(a) at least one keratolytic compound or agent,

(b) an adhesive,

(c) a dermal regeneration promoter, and

(d) a solvent.

With more particularity, the at least one keratolytic compound unclogs the pores of the skin, thereby, penetrating the dermal layer to attack the undesirable growth or wart and destroy the wart or other undesirable tissue. The preferred keratolytic compound is salicylic acid or a derivative thereof, such as a salt or ester thereof. The salicylic acid may be unsubstituted or substituted salicylic acid such as acetylsalicylic acid or the like, as well as mixtures thereof. The salicylic acid, being soluble in organic solvents, may be used herein as a powder or as a liquid. The salicylic acid or derivative thereof may be used alone or in admixture with other keratolytic compounds, such as lactic acid.

In the practice of the present invention, a particularly preferred keratolytic compound for use herein is a mixture of the salicylic acid and lactic acid.

Where a mixture of keratolytic compound e.g. salicylic acid and lactic acid, is used it will be present in a respective weight ratio of salicylic acid to other keratolytic compound of about 2:1 to 4:1 and, preferably, of about 3:1.

In preparing the present composition the keratolytic compound will be present in an amount ranging from about 12 to about 20 percent, by weight, based on the total weight.

The second component of the present composition, the adhesive, is, preferably, a non-toxic liquid acrylic or polyacrylic vehicle which acts as an adhesive for adhering the keratolytic compound to the location to be treated When it air dries on the treated area it covers the treated area during the treatment period.

Generally, as noted, the adhesive vehicle used herein is either a copolymer of methacrylic acid and methyl methacrylate or methyl methacrylate, alone. Where used, the copolymer usually, comprises a 1:1 to 1:5 volumetric ratio of methacrylic acid to the methyl methacrylate in the copolymer. Such copolymers are well known and commercially available and are, generally, described as polyacrylic acid derivatives. Methyl methacrylate is a well-known and commercially available product.

Other types of adhesives for carrying or delivering the keratolytic compound include, which may be used herein, for example, nitrocellulose, polyester polyurethanes, polyamides, polyesters, and the like, as well as mixtures thereof.

Ordinarily, the adhesive will comprise from about 5.0 to about 10.0 percent, by weight, of the total composition.

Typically, a transport medium or solvent is employed for dispersing the keratolytic compound(s) as well as for solubilizing the adhesive. Typical of these solvents include both alkanols and ketones, such as, for example, isopropyl alcohol, ethanol, acetone, methyl ethyl ketone and the like as well as mixtures thereof.

Preferably, in the practice of the present invention, a mixture of ethanol and acetone is employed as the solvent. Acetone is employed because of its low flash point enabling rapid drying of the composition. Preferably, the mixture of ethanol and acetone is used in a 1:1 weight ratio.

The solvent mixture will generally be present in the composition in an amount ranging from about 60 percent to about 85 percent, by weight, based on the total weight of the composition.

Furthermore, and as noted hereinabove, the present wart removal composition includes a skin regenerator or dermal regeneration promoter. Preferably, the regenerator used herein is L-carnitine. L-carnitine is a naturally occurring substance which resembles an amino acid and is typically described as being useful in promoting the burning of fat as well as improving heart and liver health by preventing fatty build-ups.

The dermal regenerator will be present in an amount ranging from about 0.1 to about 5 percent and preferably, from about 1.0 to 4.0 percent, by weight, based on the total weight.

If desired a minor amount of a non-reactive perfume such as a lavender oil may be used herein. Where used, the perfume or odorant will comprise no more than about one percent, by weight, of the total composition.

It is to be appreciated that the present composition avoids the use of a collodian vehicle. Rather, it provides a liquid, non-aqueous composition which is applied directly to the wart whereby the keratolytic action of the keratolytic compounds results in the mechanical removal of the epidermal cells infected with the wart viruses.

In manufacturing the present composition, the ingredients are mixed together at ambient conditions and stored in any suitable receptacle, such as an amber bottle, blue bottle, clear bottle, or the like. The composition is storage stable. To the extent any of the ingredients may precipitate out of solution, the composition is readily re-solubilized by merely shaking or otherwise agitating the components hereof, the solid ingredients, where used, readily go back into solution for topical application to the appropriate area of the user's skin.

It is to be further appreciated that the present invention may be deployed as an impregnant for a bandage or gauze which when applied over the treatment sight covers or occludes the site to further promote percutaneous absorption.

Generally, though, the composition is applied by merely daubing it onto the area to be treated and it remains thereat. As the acetone and alcohol evaporate the adhesive will cover the wound until healed and is adsorbed onto the skin directly at the wound site.

Any residual adhesive about the wound on the skin can be readily removed with alcohol or other organic solvent or can be peeled off.

Typically, the composition is deposited at the site to be treated on a daily basis for anywhere from about seven days to about thirty days. The adhesive, while covering the wound, does not act as a barrier for subsequent applications because of the solvent.

After the solvent evaporates, a white pasty growth removal residual composition is left at the site.

For a more complete understanding of the present invention, reference is made to the following non-limiting illustrative example. In the following example, all parts are by weight absent indications to the contrary.

### EXAMPLE

Into a suitable container equipped with agitation means and at room temperature is added 15 parts of salicylic acid powder, 5.0 parts of lactic acid, 8.5 parts of methyl methacrylate adhesive; and 3.5 parts of L-carnitine; 34.0 parts of ethanol 200; and 34.0 parts of acetone. The mixture is stirred until all of the ingredients go into solution.

Thereafter, agitation is halted and the composition is ready for use.

Having thus described the invention, what is claimed:

1. A topical non-aqueous liquid composition for the removal of fibroids, and papillomas, consisting essentially of:
(a) at least one keratalytic compound,
(b) an adhesive,
(c) a dermal regeneration promoter, and
(d) a solvent

2. The composition of claim 1 wherein:
(a) the at least one keratalytic compound is selected from the group consisting of salicylic acid, a salt of salicylic acid, an ester of salicylic acid, acetylsalicylic and mixtures thereof,
(b) the adhesive is selected from the group consisting of an acrylate, a polyacrylatic, nitrocellulose, polyester polyurethane, a polyamide, a polyester and mixtures thereof,
(c) the dermal regeneration promoter is L-carnitine, and
(d) the solvent is selected from the group consisting of alkanols, ketones, and mixtures thereof.

3. The composition of claim 2 which comprises:
(a) from about 12 to about 20 percent, by weight, of the at least one keratolytic compound,
(b) from about 5 to about 10 percent, by weight of adhesive, from about 0.1 to about 5 percent, by weight, of the regenerator, and
(c) from about 60 to about 85 percent, by weight, of the solvent.

4. The composition of claim 3 wherein:
(a) the keratolytic compound is a mixture of salicylic acid and lactic acid,
(b) the adhesive is methylmethacrylate, and
(c) the solvent is a mixture of an alkanol and a ketone.

5. The composition of claim 4 wherein:
the solvent is a mixture of ethanol and acetone.

6. The composition of claim 5 wherein:
(a) the salicylic acid and lactic acid are present in a respective weight ratio of about 2:1 to about 4:1, and
(b) the ethanol and acetone are present in a respective weight ratio of about 1:1.

7. The composition of claim 1 which further comprises:
a minor amount of a perfume.

8. A method for removing a fibroid or papilloma or other undesirable skin growth, comprising:
applying to the growth, the composition of claim 6.

9. The method of claim 8 which further comprises:
applying the composition to the growth on a daily basis for a period of at least about seven days.

10. The method of claim 9 which further comprises:
storing the composition in a blue bottle, and applying the composition from the bottle.

11. The method of claim 9 which further comprises:
(a) impregnating a bandage with a sufficient amount of the composition,
(b) applying the bandage to the growth,
(c) removing the bandage from the growth area after about one day,
(d) applying a new impregnated bandage to the growth site and
(e) repeating steps (b)-(d) on a daily basis for a period of about at least seven days.
